(19) United States
(12) Patent Application Publication
(10) Pub. No.: US 2019/0321493 A1
(43) Pub. Date: Oct. 24, 2019

(54) MATERIALS AND METHODS FOR TREATING REGIONAL PAIN

(71) Applicant: Mayo Foundation for Medical Education and Research, Rochester, MN (US)

(72) Inventors: Andreas S. Beutler, Rochester, MN (US); Timothy P. Maus, Rochester, MN (US); Jeff R. Anderson, Rochester, MN (US); Mark Daniel Unger, Jr., Georgetown, MA (US)

(73) Assignee: Mayo Foundation for Medical Education and Research, Rochester, MN (US)

(21) Appl. No.: 16/461,984

(22) PCT Filed: Nov. 17, 2017
(86) PCT No.: PCT/US2017/062390

§ 371 Date: May 17, 2019

Related U.S. Application Data
(60) Provisional application No. 62/424,082, filed on Nov. 18, 2016.

(51) Int. Cl. A61K 49/10; A61K 31/55; A61K 33/24; A61K 47/69; A61K 9/19; A61K 9/00; A61K 31/357; A61P 29/00; A61K 48/00

(52) U.S. Cl. CPC A61K 49/106; A61K 31/55; A61K 33/24; A61K 47/6951; A61K 48/0083; A61K 9/0019; A61K 31/357; A61P 29/00; A61K 9/19

(57) ABSTRACT

This document provides materials and methods for treating regional pain. For example, compositions including one or more analgesics can be selectively administered (e.g., by image-guided injection) to one or more nerves to treat a mammal having regional pain.

### CROSS-REFERENCE TO RELATED APPLICATIONS

This application claims the benefit of U.S. Provisional Application Ser. No. 62/424,082, filed Nov. 18, 2016. The disclosure of the prior application is considered part of (and is incorporated by reference in) the disclosure of this application.

### BACKGROUND

### 1. Technical Field

This document relates to materials and methods for treating regional pain. For example, compositions including one or more analgesics can be selectively administered (e.g., by image-guided injection) to one or more nerves to treat a mammal having regional pain.

### 2. Background Information

Regional pain is a major health problem that greatly affects quality of life. Regional pain can be of limited duration or chronic such as that related to complex regional pain syndrome. Regional pain can present as a symptom of numerous diseases, but may also present as a lasting consequence of injury or damage to muscle, joints, ligaments, skin, inner organs, and/or nerves. Pain management options for regional pain include the use of systemic drugs and steroid injections as well as surgical interventions. Opioid drugs are often prescribed for regional pain management. Systemic opioid drugs that are delivered orally or intravenously are highly habit forming and their increased use has contributed to what has been considered an epidemic of drug abuse (Manchikanti et al., 2012 Pain Physician 15(3 Suppl):ES9-38). Furthermore, individuals can develop a tolerance to systemic opioid drugs decreasing or removing their efficacy for pain management. Steroid injections are sometimes ineffective and questions have arisen regarding the reproducibility of effective response that appears to be influenced by a myriad of factors (Koes et al., 1995 Pain 63:279-288; and MacVicar et al., 2012 Pain Medicine 14:14-28). Currently, there is no cure for regional pain.

### SUMMARY

This document provides materials and methods for administering compositions including one or more analgesics to treat a mammal having regional pain. For example, this document provides materials and methods for selectively administering (e.g., by image-guided injection) compositions including one or more analgesics to one or more neural tissues to reduce and/or treat regional pain.

As demonstrated herein, image-guided injection can be used to deliver compositions including an analgesic and a contrast agent into a precise anatomical location (e.g., a dorsal root ganglion (DRG)) to treat regional pain. For example, a composition including resiniferatoxin (RTX) and gadoteridol can be administered by magnetic resonance imaging (MRI) guided intraganglionic (IG) injection to the DRG to induce neurolysis of the DRG. A contrast agent allows one to visualize both the anatomical location of the needle and the administration of the composition, and enables one to predict successful delivery of the composition to the desired anatomical location during the procedure.

In general, one aspect of this document features a composition comprising an analgesic and an imaging agent. The analgesic can be a transient vanilloid receptor 1 (TRPV1) agonist (e.g., RTX, tinyatoxin, capsaicin, and derivatives and/or analogs thereof). For example, the TRPV1 agonist can be RTX. The analgesic can be a TRPV1 antagonist (e.g., capsazepine, ruthenium red, and derivatives and/or analogs thereof). The analgesic can be a nucleic acid encoding a polypeptide useful for treating pain. The nucleic acid encoding a polypeptide useful for treating pain can be present in a delivery vehicle. The delivery vehicle can be an adeno-associated virus vector. The imaging agent can be a non-neurotoxic imaging agent. The imaging agent can include gadolinium. The composition also can include a solubilizer. The solubilizer can be a non-neurotoxic solubilizer. The solubilizer can be a cyclodextrin (e.g., sulfobutyl ether β-cyclodextrin). The composition can be in the form of a pellet, gel, or lyophilized powder.

In another aspect, this document features a method for treating regional pain in a mammal. In some cases, the method includes, or consists essentially of, injecting a composition including an analgesic and an imaging agent to a neural tissue of a mammal identified as having regional pain, where the regional pain is reduced. In some cases, the method includes, or consists essentially of, injecting a composition including an analgesic and an imaging agent to a neural tissue of a mammal identified as having regional pain, where neurolysis of the neural tissue is induced. The method mammal can be a human. The injection can include a spinal injection route (e.g., an IG injection or an injection to the subarachnoid space). For example, the spinal injection route can be an IG injection. The neural tissue can be a ganglion (e.g., a dorsal root ganglion). The methods also can include monitoring the injection. The monitoring can include an imaging technique (e.g., ultrasound, radiography, X-ray, computed tomography (CT), fluoroscopy, positron emission tomography, and MRI). For example, the imaging technique can be MRI. The composition can include from about 10 μL to about 500 μL (e.g., about 100 μL).

In another aspect, this document features a kit. A kit can include an analgesic, an imaging agent, a non-neurotoxic solubilizer, a guide needle, and a delivery needle. The kit of analgesic can be lyophilized resiniferatoxin. The non-neurotoxic solubilizer can be a cyclodextrin (e.g., sulfobutyl ether β-cyclodextrin).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1D are fluorescent immunohistochemistry (IHC) images. FIGS. 1A and 1B show normal levels of nociceptive fibers (FIG. 1A) and elimination of nociceptive fibers following intraganglionic injection of RTX solubilized in water with sulfobutyl ether β-cyclodextrin (FIG. 1B) in the dorsal horn of the spinal cord (demarcated by white line). FIGS. 1C and 1D show normal levels of nociceptive fibers (FIG. 1C) and elimination of nociceptive fibers following intraganglionic injection of RTX solubilized in water with sulfobutyl ether β-cyclodextrin (FIG. 1D) in the dorsal root ganglion (demarcated by white line). Nociceptive fibers in all panels are visualized using the biomarker substance P (sP).

FIGS. 2A-2C are fluorescent IHC images of a DRG. FIG. 2A is a DRG from a control animal shows non-nociceptive neurons and non-neuronal cells, and nociceptive neurons (stained for sP). FIG. 2B is a DRG after successful magnetic resonance imaging (MRI)-guided injection of RTX (solubilized in water with sulfobutyl ether β-cyclodextrin) demonstrating elimination of most nociceptive neurons (absence of sP fluorescence). FIG. 2C is a DRG after failed RI-guided injection of RTX (solubilized in water with sulfobutyl ether β-cyclodextrin) showing normal numbers of nociceptive neurons visualized as fluorescence (sP is retained). The outcomes in FIG. 2B and FIG. 2C were predicted from intraprocedural imaging performed during RTX delivery (noted as a success (B) and failure (C) to achieve the intended contrast media dispersion).

FIGS. 3A and 3B contain IHC images showing normal levels of nociceptive fibers (3A) and elimination of nociceptive fibers following periganglionic (epidural) injection of RTX solubilized in water with sulfobutyl ether β-cyclodextrin (3B) in the dorsal horn of the spinal cord (demarcated by white line).

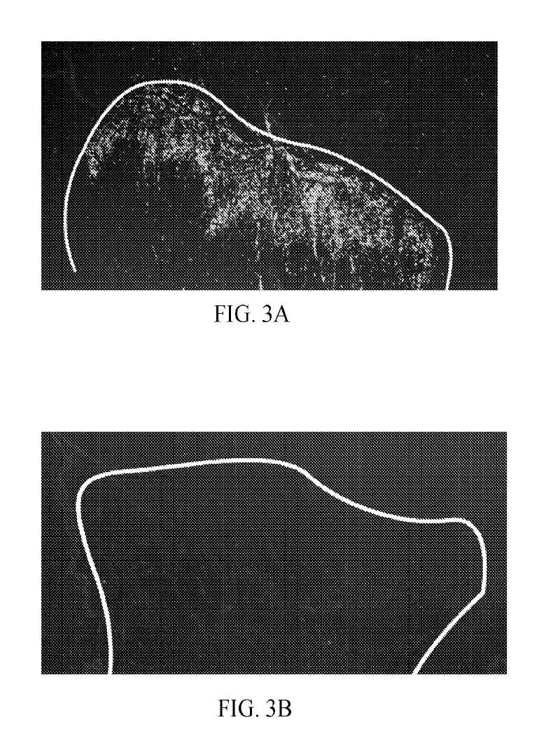

FIGS. 4A and 4B contain MRI images of IG co-injection of a contrast agent and RTX solubilized in water with sulfobutyl ether β-cyclodextrin (RTXcap) to a DRG. FIG. 4A is a DRG that was successfully targeted (shown by the concentrated contrast) in the anatomic DRG following injection. FIG. 4B is a DRG that was unsuccessfully targeted (shown by the dispersed, minimal contrast) despite Mill appearing to show correct needle location.

FIGS. 5A-5C illustrate drug distribution in swine DRG. FIG. 5Q is a schematic of convection enhanced delivery (CED) needle placement in a DRG. FIG. 5B contains an image of a control DRG. FIG. 5C contains an image of a 4′,6-diamidino-2-phenylindole (DAPI) injected DRG.

FIGS. 6A-6E shows that RTXcap induced lysis of nociceptive sensory neurons in the swine DRG. RTXcap (500 ng) was delivered by IG CED. FIGS. 6A-6D contain immunohistochemistry images for sP. FIG. 6A contains an image of a DRG control (contralateral). FIG. 6B contains an image of a DRG after successful RTXcap injection. FIG. 6C contains an image of a spinal cord (SC) control (post. horn). FIG. 6D contains an image of a SC after successful RTXcap treatment of ipsilateral DRG (corresponding area to C). FIG. 6E contains a graph of the fraction of sP+ cells (n=5 swine).

FIG. 7 contains graphs showing analgesia after IG CED delivery of RTXcap demonstrated by behavior testing. Black: RTXcap. Gray: control (inactive vehicle). Open symbols: Animals treated in an “open label” experiment (cohort 1). Closed symbols: Animals treated in a randomized, blinded experiment (cohort 2).

### DETAILED DESCRIPTION

This document provides materials and methods for treating regional pain. For example, this document provides materials and methods for administering compositions including one or more analgesics to treat a mammal having regional pain. In some cases, a composition including one or more analgesics can be selectively administered (e.g., by image-guided injection) to one or more target tissues (e.g., nerves) to treat a mammal having regional pain. The materials and methods provided herein can be used to reduce regional pain. For example, a composition including one or more analgesics can be used to induce neurolysis (e.g., targeted neurolysis). For example, a composition including one or more analgesics can be administered to a DRG to induce targeted neurolysis resulting in neuronal cell death of one or more cells within the DRG.

A composition including one or more (e.g., 2, 3, 4, 5, 6, 7, 8, 9, 10, or more) analgesics described herein can include any appropriate analgesic. As used herein, an “analgesic” is any agent that reduces nociception and/or pain. An analgesic can be any appropriate type of molecule (e.g., chemical compounds, small molecules, biologics such as polypeptides (e.g., antibodies) and/or nucleic acids). Reducing nociception and/or pain can be achieved by any appropriate mechanism. An analgesic can reduce nociception and/or pain by decreasing nociceptive signaling. An analgesic can reduce nociception and/or pain by reducing inflammation and/or glial activation. An analgesic can reduce nociception and/or pain by neurolysis of nociceptive neurons. In some cases, an analgesic can reduce nociception and/or pain by an indirect mechanism (e.g., by maintaining or restoring normal nerve function to treat or prevent a peripheral nerve condition such as neuropathy (including, for example, hereditary neuropathy, diabetic neuropathy, and neuropathy)). An analgesic can be a non-opioid analgesic. An analgesic can be naturally occurring or synthetic. In some cases, an analgesic can be a transient vanilloid receptor 1 (TRPV1) agonist. A TRPV1 agonist can irreversibly open the TRPV1 channel causing the channel to become permeable to cations (e.g., calcium); the influx of cations leads to neurolysis of the TRPV1-expressing neuron, resulting in alleviation of pain. Examples of TRPV1 agonists include, RTX, tinyatoxin, capsaicin, 12-deoxyphorbol 13-phenylacetate 20-homovanillate, anadamide, and N-Oleoydopamine, and derivatives and/or analogs thereof. In some cases, an analgesic can be a TRPV1 antagonist. A TRPV1 antagonist can block TRPV1 activity, thus reducing pain. A TRPV1 antagonist can be a competitive antagonist or a non-competitive antagonist. Examples of TRPV1 antagonists include, capsazepine, ruthenium red, 5-Iodoresiniferatoxin, A425619, and A784168, and derivatives and/or analogs thereof. In some cases, an analgesic can be a nucleic acid and/or a polypeptide (e.g., a nucleic acid encoding a polypeptide useful for treating pain, a nucleic acid transcribing an RNA useful for treating pain, and/or a nucleic acid encoding a polypeptide useful for treating pain). Examples of polypeptides useful for treating pain include, without limitation, enkephalin (e.g., pre-pro-enkephalin), β-endorphin (e.g., pre-pro-β-endorphin), interleukin 10 (IL-10), glutamic acid decarboxylase (GAD), endomorphin 1, and endomorphin 2. Additional examples of polypeptides useful for treating pain include those described elsewhere (see, e.g., Pleticha et al., 2016 Mayo Clinic Proceedings 91:522-533; Storek et al., 2008 PNAS 105:1055-1060; and Pleticha et al., 2015 Gene Therapy 22:202-208).

An analgesic described herein can be administered using any appropriate delivery vehicle. For example, in cases where an analgesic is a nucleic acid encoding a polypeptide useful for treating pain, the nucleic acid can be incorporated into a delivery vehicle that can drive expression of the nucleic acid. Examples of delivery vehicles include, without limitation, non-viral vectors (e.g., plasmids (e.g., expression plasmids), liposomes, and polymersomes) and viral vectors (e.g., adeno-associated virus (AAV) vectors, HSV vectors, and lentiviral vectors). For example, a nucleic acid encoding a polypeptide useful for treating pain can be delivered using an AAV (e.g., AAV serotype 1) vector.

In some cases, a composition including one or more analgesics described herein also can include one or more imaging agents. An imaging agent can be non-neurotoxic. An imaging agent can be a contrast agent. An imaging agent can be a fluorescent agent. An imaging agent can be a linear molecule or a caged molecule. Examples of imaging agents include, without limitation, microbubbles, ionic iodinated compounds (e.g., diatrizoate, metrizoate, iothalamate, ioxaglate), non-ionic iodinated compounds (e.g., iopamidol, iohexol, ioxilan, iopromide, iodixanol, ioversol), barium containing compounds (e.g., barium sulfate), radio-translucent gases (e.g., air, carbon dioxide), radiotracers containing the radionuclide carbon-11 (e.g., [11C] carbon dioxide), radiotracers containing the radionuclide nitrogen-13, radiotracers containing the radionuclide oxygen-15 (e.g., [15O] water), radiotracers containing the radionuclide fluorine-18 (e.g., [18F] fluoride, [18F] fluorodeoxyglucose, [18F] fluorothymidine, [18F] fluoromisonidazole), radiotracers containing the 64Cu-ATSM: 64Cu diacetyl-bis(N4-methylthiosemicarbazone), radiotracers containing the radionuclide gallium-68 (e.g., [68Ga] gallium DOTATOC and [68Ga] galium DOTATATE), radiotracers containing the radionuclide zirconium-89, radiotracers containing the radionuclide rubidium-82, radiotracers containing technetium-99m (e.g., [99mTc] technetium medronic acid, [99mTc] technetium sestamibi), radiotracers containing thallium-201m, nanoparticles (e.g., iron oxide nanoparticles, silver nanoparticles, gold nanoparticles, iron platinum nanoparticles), manganese ions, ionic gadolinium containing compounds (e.g., gadopentetic acid, gadobenate dimeglumine, gadoteric acid, gadoterate meglumine, gadoxetate disodium, gadofosveset trisodium), and/or non-ionic gadolinium containing compounds (e.g., gadodiamide, gadoversetamide, gadoteridol, gadobutrol). For example, a composition including one or more analgesics described herein also can include a gadolinium-based contrast agent. In some cases, a composition described herein can include an imaging agent at between about 0.1% and about 2.5% volume/volume solution (e.g., between about 0.2% and about 2%, between about 0.5% and about 1.7%, between about 0.7% and about 1.5%, or between about 0.9% and about 1.2% volume/volume solution) based on about a 0.5 M stock solution preparation of the imaging agent. For example, a composition described herein can include an imaging agent at about 1% volume/volume solution.

In some cases, a composition including one or more analgesics described herein also can include any appropriate solubilizer. A solubilizer can be a non-neurotoxic solubilizer. A solubilizer can be a cyclodextrin. Examples of cyclodextrins include, without limitation, Captisol®, α-cyclodextrin, β-cyclodextrin, and sulfobutyl ether β-cyclodextrin.

In some cases, a composition including one or more analgesics described herein also can include any appropriate solvent. A solvent can be a non-organic solvent. A solvent can be a non-neurotoxic solvent. Examples of solvents include, without limitation, water, saline, ethanol, DMSO, and phosphate buffered saline (PBS). For example, a composition including one or more analgesics described herein also can include (e.g., be reconstituted in) water and/or saline.

In some cases, a composition including one or more analgesics described herein does not include any organic solvent (e.g., a neurotoxic organic solvent). Examples of neurotoxic organic solvents include, without limitation, acetone, benzene, chloroform, dimethyl sulfoxide, ethanol, and hexane.

A composition including one or more analgesics described herein can be in any appropriate form. A composition can be, for example, a solution, a suspension, a gel, a pellet, or a powder (e.g., lyophilized powder). A composition described herein can be dissolvable (e.g., in vivo). A composition described herein can exhibit controlled (e.g., time delayed) and/or sustained release of one or more analgesics. In cases where a composition described herein is in the form of a pellet, the pellet can be coated with an analgesic, the pellet can encapsulate an analgesic, and/or the analgesic can be dispersed throughout the pellet. In cases where a composition described herein is in the form of a pellet, the pellet can be a nanoparticle. For example, a pellet can be between about 0.1 mm and about 10 mm in size (e.g., between about 0.1 mm and about 8 mm, between about 0.2 mm and about 5 mm, between about 0.3 mm and about 3, between about 0.4 mm and about 2 mm, or between about 0.5 mm and about 1 mm in diameter). For example, a pellet including one or more analgesics described herein can be about 7 mm in diameter. A nanoparticle can be any appropriate shape (e.g., spheroid or non-spheroid (e.g., cylindrical or conical)). In cases where a composition described herein is in the form of a gel, the gel can be a polymer based gel.

A composition including one or more analgesics described herein can include one or more pharmaceutically acceptable carriers (additives) and/or diluents. Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

This document also provides methods of treating regional pain. When treating a regional pain as described herein, the regional pain can be any appropriate regional pain. Regional pain can be chronic or acute. Regional pain can be intermittent or constant. Regional pain can be frequent or infrequent. Regional pain can be present in any region of a body. Regional pain can be present in one or more (e.g., 2, 3, 4, 5, 6, 7, 8, 9, 10, or more) regions of the body. Examples of body regions that can exhibit regional pain and can be treated with the materials and methods described herein include, without limitation, finger, finger joint, thumb, thumb joint, hand, wrist, wrist joint, arm, elbow, elbow joint, shoulder, shoulder joint, toe, toe joint, foot, ankle, ankle joint, leg, knee, knee joint, hip, hip joint, genitals, torso, abdomen, chest, back, lower back, middle back, upper back, and/or neck. Regional pain can be present in any appropriate body tissue. Examples of body tissues that can exhibit regional pain and can be treated with the materials and methods described herein include, without limitation, bone, muscular, cartilage, tendon, ligament, skin, inner organs, and/or nerves. Regional pain can be related to (e.g., a symptom of) a syndrome, disease, or disease state. Examples of syndromes and/or diseases that can exhibit regional pain and can be treated with the materials and methods described herein include, without limitation, chronic axial back pain, chronic joint pain (e.g. due to osteoarthritis or inflammatory states such as spondyloarthropathy), radicular pain (with or without radiculopathy), neuropathy, type I complex regional pain syndrome, type II complex regional pain syndrome, reflex sympathetic dystrophy syndrome, reflex neurovascular dystrophy, anterior cutaneous nerve entrapment syndrome, bursitis, prostatitis, chronic pelvic pain syndrome, chronic wound pain, degenerative disc disease, failed back syndrome, hand-arm vibration syndrome, interstitial cystitis, lateral epicondylitis, post-vasectomy pain syndrome, sickle-cell disease, chronic tendinitis, and/or vulvodynia.

Any type of mammal having regional pain can be treated as described herein. For example, humans and other primates such as monkeys having regional pain can be treated with a composition including one or more analgesics as described herein. In some cases, dogs, cats, horses, cows, pigs, sheep, rabbits, mice, and rats can be treated with a composition including one or more analgesics as described herein.

Methods for treating a mammal (e.g., a human) having regional pain can include identifying the mammal as having regional pain or as being at risk of developing regional pain. Any appropriate method can be used to identify a mammal having regional pain or at being at risk for developing regional pain. In some cases, a mammal having regional pain or at risk of developing regional pain can be diagnosed by a medical professional (e.g., a medical professional experienced in the diagnosis of pain syndromes and/or disorders of the peripheral nervous system such as anesthesiologists, neurologists, orthopedists, neurosurgeons, physiatrists, radiologists, and interventional radiologists).

Once identified as having regional pain or as being at risk for developing regional pain, the mammal can be administered a composition including one or more analgesics described herein. A composition including one or more analgesics can be administered to a mammal having or at risk of developing regional pain by any appropriate injection technique (e.g., with the use of a convection enhanced delivery (CED) needle). In some cases, the injection can be an image-guided injection, where an imaging technique is used to visualize the placement of one or more injection needles. Imaging techniques that can be used in image-guided injection include, without limitation, ultrasound, radiography, X-ray, CT (e.g., single-photon emission CT), fluoroscopy, positron emission tomography, and MRI. A composition described herein can be injected by any appropriate route. In some cases, an injection route is a spinal injection route. Examples of injection routes that can be used to administer a composition described herein include, without limitation, IG injection, and injection to the periganglionic subarachnoid space. In some cases, a composition described herein can be used to target (e.g., injected into or nearby) any appropriate tissue. In some cases, a tissue can be a neural tissue. A neural tissue can be targeted by injecting a composition described herein into the neural tissue and/or nearby the neural tissue. Examples of neural tissues into which a composition described herein can be injected include, without limitation, ganglia (e.g., the DRG), spinal nerve, preganglionic fibers, and paraganglia. Examples of neural tissues nearby which a composition described herein can be injected include, without limitation, the periganglionic subarachnoid space. In some cases, a composition described herein can be injected in a small volume. For example, a composition described herein can be injected in about from about 10 μL to about 500 μL (e.g., about 15 μL to about 400 μL, about 20 μL to about 350 μL, about 25 μL to about 300 μL, about 50 μL to about 250 μL, about 75 μL to about 200 μL, or about 90 μL to about 150 μL). For example, an effective amount of RTX can be about 100 μL.

A composition including one or more analgesics described herein can be administered at any appropriate time. In some cases, a composition including one or more analgesics described herein can be administered prophylactically. For example, a composition including one or more analgesics described herein can be administered to prevent the development of pain (e.g., at the conclusion of a standard spine operation). In some cases, a composition including one or more analgesics described herein can be administered therapeutically. For example, a composition including one or more analgesics described herein can be administered to treat pain.

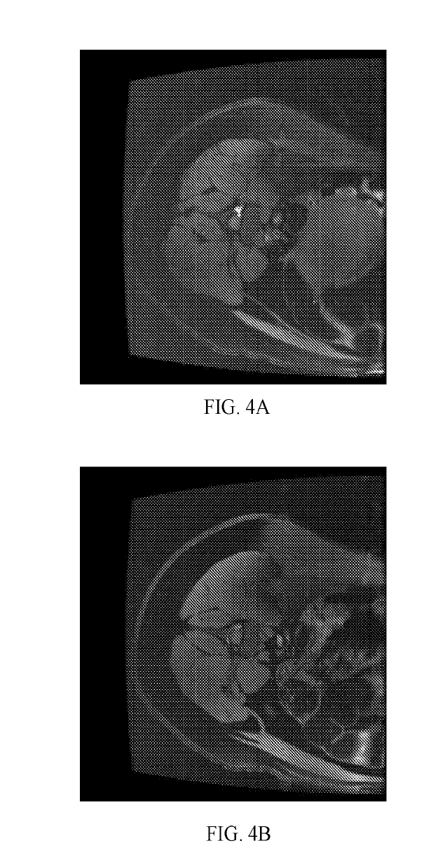

In cases where a composition includes an imaging agent, the methods described herein also can include monitoring (e.g., real-time monitoring) the injection of the composition using one or more imaging techniques. In some cases, an analgesic and an imaging agent can be administered together (e.g., co-injected as a single composition). In some cases, an analgesic and an imaging agent can be administered separately (e.g., injected independently of one another where the imaging agent can be administered prior to, concurrent with, or after the analgesic). Images obtained by an imaging technique can be obtained intermittently or continuously. Imaging techniques that can be used to monitor the injection of the composition include, without limitation, ultrasound, radiography, X-ray, CT (e.g., single-photon emission CT), fluoroscopy, positron emission tomography, and MRI. Image-guided injection techniques can be performed as described elsewhere (see, e.g., Pleticha et al., 2013 J. Neurosci. Meth. 216:10-5; and Pleticha et al., 2014 J Neurosurg 121:851-858). The anatomical localization of the injection needle can be insufficient to ensure successful delivery of the therapeutic agent into a target tissue (see, e.g., FIG. 4). In some cases, monitoring of the injected composition during the procedure can provide real-time feedback that can be used to predict success or failure of the injection. In some cases, monitoring of an injection can allow for real-time adjustments to the injection procedure.

Effective doses can vary depending on the severity of the regional pain, the age and general health condition of the subject, excipient usage, the possibility of co-usage with other therapeutic treatments such as use of other agents, and the judgment of the treating physician.

An effective amount of a composition containing one or more analgesics described herein can be any amount that reduces regional pain without producing significant toxicity to the mammal. Pain can be evaluated using any appropriate method. For example, pain can be evaluated by a medical professional (e.g., a medical professional experienced in the diagnosis of pain syndromes and/or disorders of the peripheral nervous system such as anesthesiologists, neurologists, orthopedists, and neurosurgeons, physiatrists, radiologists, and interventional radiologists). In some cases, composition described herein contains a high concentration of an analgesic described herein. For example, an effective amount of an analgesic such as RTX (e.g., RTX administered via periganglionic (epidural) injection) can be from about 0.05 μg to about 50 μg (e.g., about 0.08 μg to about 40 μg, about 0.1 μg to about 30 μg, about 0.15 μg to about 25 μg, about 0.2 μg to about 20 μg, about 0.5 μg to about 15 μg, about 1 μg to about 10 μg, or about 2.5 μg to about 8 μg). For example, an effective amount of an analgesic such as RTX (e.g., RTX administered via intraganglionic (IG) injection) can be from about 0.005 μg to about 10 μg (e.g., about 0.01 μg to about 8 μg, about 0.05 μg to about 6 μg, about 0.1 μg to about 4 μg, about 0.3 μg to about 3 μg, about 0.4 μg to about 1 μg). In some cases, an effective amount of RTX can be about 5 μg. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, and severity of the regional pain may require an increase or decrease in the actual effective amount administered.

A composition including one or more analgesics described herein can be administered to a mammal having regional pain as a combination therapy with one or more additional agents/therapies used to treat regional pain. For example, a combination therapy used to treat regional pain can include administering to the mammal (e.g., a human) a composition including one or more analgesics and one or more pain treating agents such as pain relievers (e.g., aspirin, ibuprofen, and naproxen), anti-inflammatories, steroids (e.g., corticosteroids), systemic opioids (e.g., morphine, and oxycodone) and/or low dose systemic opiods (e.g., morphine, and oxycodone). For example, a combination therapy used to treat regional pain can include administering to the mammal (e.g., a human) a composition including one or more analgesics and one or more pain treating therapy such as laminectomy, decompression, spinal fusion, and/or neural stimulator implantation. In cases where one or more analgesics are used in combination with one or more additional agents/therapies to treat pain, the one or more additional agents/therapies can be administered at the same time or independently. For example, the composition including one or more analgesics can be administered first, and the one or more additional agents/therapies can be administered second, or vice versa.

This document also provides kits. In some cases, a kit can include one or more analgesics described herein and one or more imaging agents described herein. For example, kit can include a lyophilized composition including one or more analgesics (e.g., lyophilized RTX powder). A kit also can include one or more additional agents (e.g., an imaging agent and/or a solubilizer such as cyclodextrin). For example, a kit can include lyophilized RTX and an imaging agent. For example, a kit can include a lyophilized RTX powder and cyclodextrin. For example, a kit can include a lyophilized RTX powder, cyclodextrin, and an imaging agent. In cases where a kit includes a lyophilized RTX powder and an additional agent (e.g., an imaging agent and/or a solubilizer), the additional agent can be lyophilized and provided together with the lyophilized RTX powder.

In some cases, a kit can include one or more delivery systems. For example, a kit can include a guide needle (e.g., a beveled guide needle having a standard or a non-standard bevel), a tunneler or trocar that fits within the guide needle, a stylet (e.g., a stylet that is stepped at the tip) that fits within the guide needle, and/or a delivery needle (e.g., stepped delivery needle). A delivery needle can have a blunt tip. A delivery needle can have a single outlet port at the tip. A delivery needle can have one or more outlet ports located on the walls of the needle shaft. In some cases, a kit can include directions for use of the kit. For example, a kit can include instructions to reconstitute a lyophilized RTX powder in water and/or saline. For example, a kit can include instructions for administering compositions described herein to a mammal (e.g., a human) having regional pain. Examples of needles and other materials that can be included in a kit for use with the materials and methods described herein can be as described elsewhere (see, e.g., Pleticha et al., 2013 J. Neurosci. Meth. 216:10-5; and Pleticha et al., 2014 J Neurosurg 121:851-858).

The invention will be further described in the following examples, which do not limit the scope of the invention described in the claims.

### EXAMPLES

### Example 1: MRI-Guided DRG-Targeted Delivery of Resiniferatoxin (RTX)

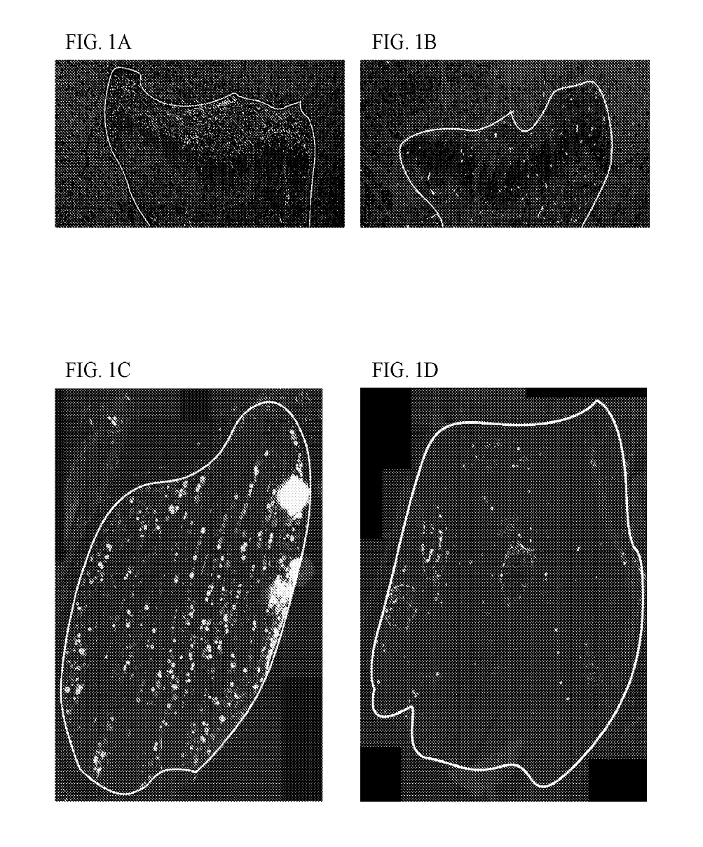

Domestic swine were chosen because the porcine lumbar spinal column resembles the human lumbosacral anatomy. Swine were sedated with Telazol and Xylazine, intubated and kept under deep isoflurane inhalation anesthesia for the duration of the procedure titrated to effect. Swine were placed in an MRI scanner (GE 3T, GE Healthcare). Overview MRI imaging was obtained of the lumbosacral spine. DRG at the L4, L5, S1, and S2 level were outlined (FIG. 1A).

The guide needle was passed through the skin lateral to the midline and incrementally advanced ventromedially toward the DRG intraprocedural MRI imaging monitored advancement of the needle, and any deviations from the optimal trajectory were corrected. When the needle tip was visualized directly adjacent to the dorsal aspect of the DRG, the stylet of the guide needle was withdrawn. The stepped stylet was then inserted through the guide needle. The length of the stepped stylet exceeded the length of the guide needle and therefore only the stepped tip of the stylet but not the Quincke tip of the guide needle penetrated the DRG parenchyma. The stepped stylet was then withdrawn and replaced by the stepped needle. The prior insertion of the stepped stylet prevented clogging of the narrow needle tip. RTX 5 μg and gadoteridol 1% V/V was injected in a volume of 50 μl per DRG over a period of 5 minutes (RTX was solubilized in water with sulfobutyl ether β-cyclodextrin, designated as RTXcap). MRI imaging verified when the injection was successful and when the injection needed to be repeated (FIG. 4). Pigs were sacrificed 5 weeks post injection and the tissues harvested for histopathological analysis.

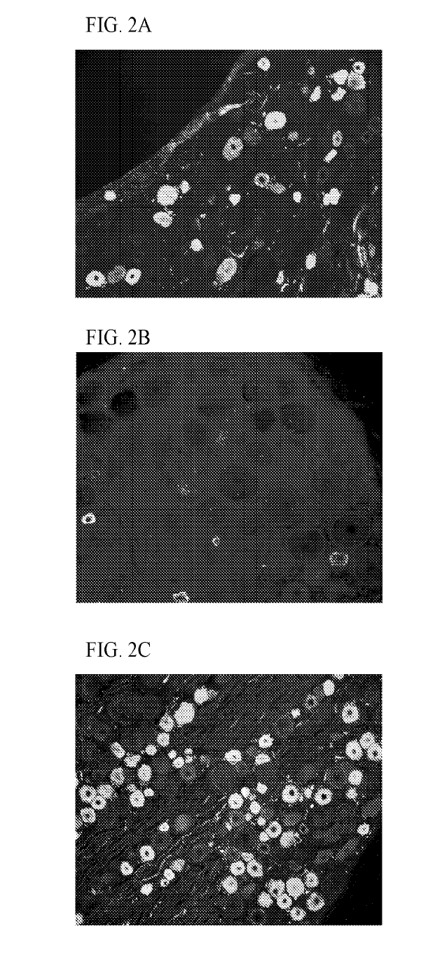

These results demonstrate that RTX delivery can result in neurolysis of the targeted tissue (see, e.g., FIG. 1 and FIG. 2).

### Example 2: CED-Guided DRG-Targeted Delivery of Resiniferatoxin (RTX)

An incision was made in the overlying skin at an anatomical level affected by pain in a patient. The subcutaneous fat and musculature were incised and reflected, exposing the bony lamina. Using appropriate instruments, a portion of the lamina was removed to expose the neural elements. The meningeal sleeve surrounding the DRG and spinal nerve was surgically exposed by microdissection of bone and tissue, which may require extending the laminectomy dissection, using standard techniques such as blunt or sharp dissection with meticulous hemostasis. Thereby the DRG was exposed and grossly visualized. A convection enhanced delivery needle was placed into the exposed DRG. RTXcap was injected in a preparation that also contains a contrast agent (e.g., in this case DAPI) allowing direct visualization of analgesic agent injection by eye sight with or without a surgical loupe or surgical operating microscope.

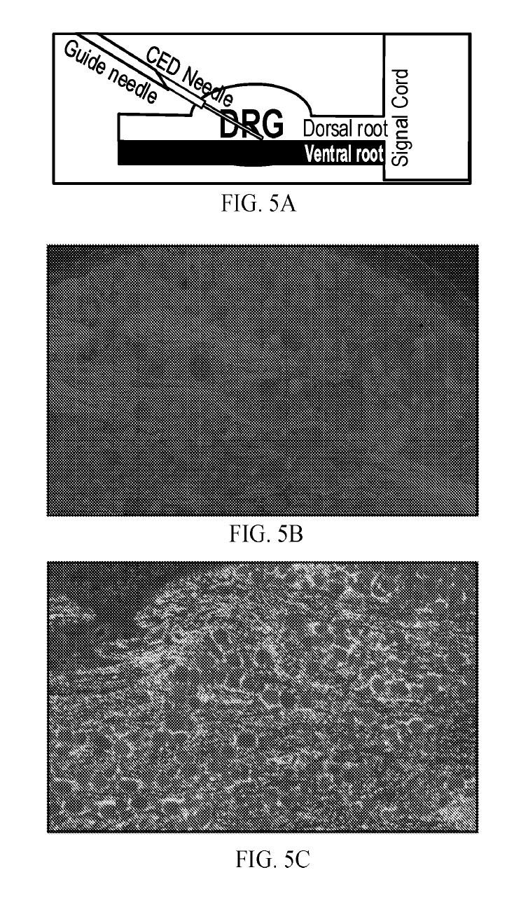

Convection enhanced delivery (CED) of DAPI demonstrated good distribution throughout the DRG (FIG. 5).

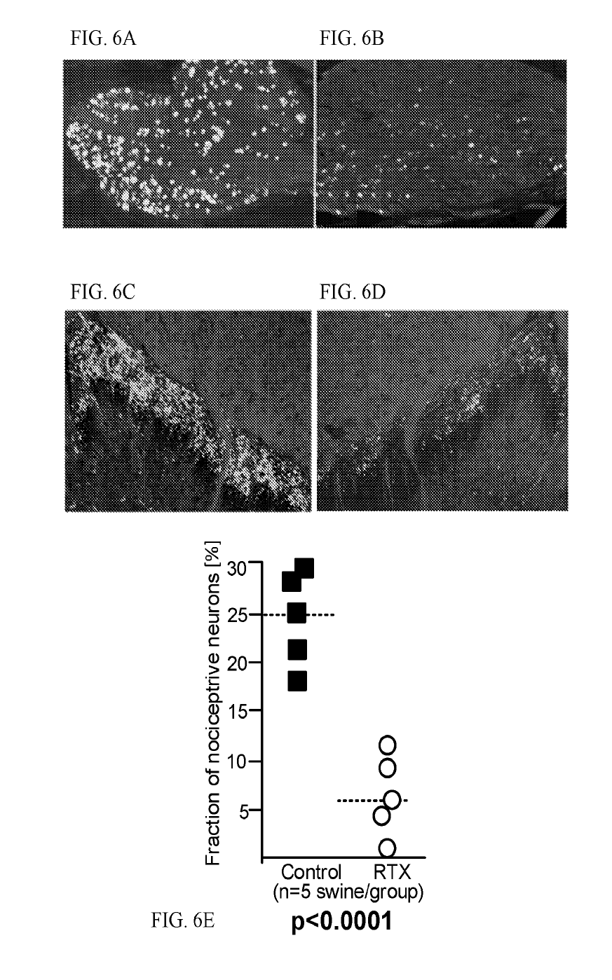

Delivery of RTXcap (500 ng) by IG CED to the DRG induced lysis of nociceptive sensory neurons in the swine DRG (FIG. 6).

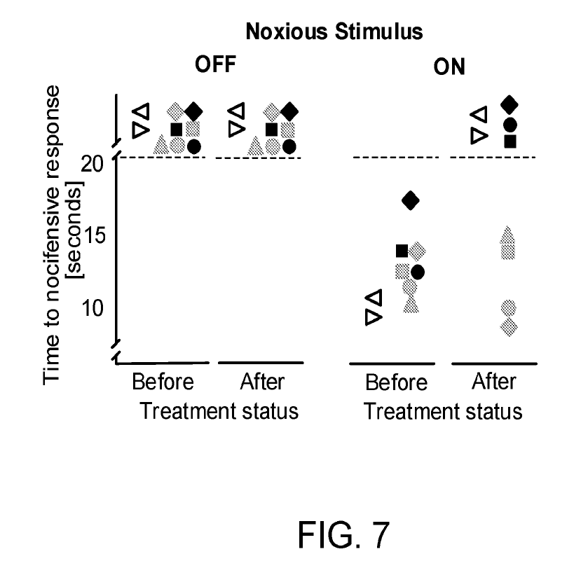

To assess the longevity of the analgesic efficacy, swine were assessed before and after treatment with spinal RTXcap or vehicle (FIG. 7). Animals were tested for nocifensive (pain-avoiding) behavior using noxious heat as a stimulus (delivered to the hind leg by a CO₂ laser). Time to limb withdrawal was measured as outcome (recorded by a custom “force place” device). In this testing paradigm animals remain motionless (>20 seconds) in the absence of noxious stimulation as seen in the left panels of FIG. 7 (“OFF”). Noxious heat leads to early limb withdrawal (“ON”), which was seen in all animals prior to treatment. After spinal drug delivery, RTXcap animals no longer responded to noxious heat, while controls remained unaffected; the difference was significant (p<0.001). Treatment was delivered at four lumbar levels by the epidural route. Laser time was limited to 20 seconds to avoid tissue injury. RTXcap provided a long-term therapeutic effect in vivo in large animals.

These results demonstrate that RTXcap delivery can result in neurolysis of the targeted tissue.

### Example 3: MRI-Guided DRG-Targeted Delivery of a Nucleic Acid Molecule

The subject is placed in an MRI scanner. Overview MRI imaging is obtained of the lumbosacral spine. The guide needle is passed through the skin lateral to the midline and incrementally advanced ventromedially toward the DRG. Intraprocedural MRI imaging monitors advancement of the needle, and any deviations from the optimal trajectory is corrected. When the needle tip is visualized directly adjacent to the dorsal aspect of the DRG, the stylet of the guide needle is withdrawn. The stepped stylet is then inserted through the guide needle. The length of the stepped stylet exceeds the length of the guide needle and therefore only the stepped tip of the stylet but not the Quincke tip of the guide needle penetrates the DRG parenchyma. The stepped stylet is then withdrawn and replaced by the stepped needle. The prior insertion of the stepped stylet prevents clogging of the narrow needle tip. A formulation consisting of phosphate buffer saline, a nucleic acid molecule in a self-complementary AAV serotype 1 virus, and the imaging agent gadoteridol are perfused into the DRG. MM imaging verifies when the injection is successful and when the injection needs to be repeated.

### Example 4: MRI-Guided DRG-Targeted Delivery of an RTX Nanoparticle

The subject is placed in an MRI scanner. Overview MRI imaging is obtained of the lumbosacral spine. The guide needle is passed through the skin lateral to the midline and incrementally advanced ventromedially toward the DRG. Real time MRI imaging monitors advancement of the needle, and any deviations from the optimal trajectory is corrected. When the needle tip is visualized directly adjacent to the dorsal aspect of the DRG, the stylet of the guide needle is withdrawn. The stepped stylet is then inserted through the guide needle. The length of the stepped stylet exceeds the length of the guide needle and therefore only the stepped tip of the stylet but not the Quincke tip of the guide needle penetrates the DRG parenchyma. RTX suspended in a sustained release nanoparticle is injected into the pocket inside the DRG created by stepped stylet.

### Example 5: DRG-Targeted Co-Delivery of AAV with an Optically Visualizable Contrast Agent in Conjunction with a Laminectomy

An incision is made in the overlying skin at an anatomical level affected by pain in a patient. The subcutaneous fat and musculature are incised and reflected, exposing the bony lamina. Using appropriate instruments, a portion of the lamina is removed to expose the neural elements. The meningeal sleeve surrounding the DRG and spinal nerve is surgically exposed by microdissection of bone and tissue, which may require extending the laminectomy dissection, using standard techniques such as blunt or sharp dissection with meticulous hemostasis. Thereby the DRG is exposed and grossly visualized. A convection enhanced delivery needle is placed into the exposed DRG. An analgesic agent in an AAV gene vector is injected in a preparation that also contains a contrast agent allowing direct visualization of analgesic agent injection by eye sight with or without a surgical loupe or surgical operating microscope.

### Example 6: DRG-Targeted Co-Delivery of RTX with a Fluorescently Visualizable Contrast Agent in Conjunction with a Decompression

An incision is made in the overlying skin at an anatomical level affected by pain in a patient. The subcutaneous fat and musculature are incised and reflected, exposing the bony lamina. Using appropriate instruments, a portion of the lamina is removed to expose the neural elements. A surgical spine procedure is performed that accesses the bones of the vertebral column. The meningeal sleeve surrounding the DRG and spinal nerve is surgically exposed by microdissection of bone and tissue, which may require extending the laminectomy dissection, using standard techniques such as blunt or sharp dissection with meticulous hemostasis. Thereby the DRG is exposed and grossly visualized. A convection enhanced delivery needle is placed into the exposed DRG. The analgesic agent, RTX, is injected in a preparation that also contains a contrast agent allowing visualization of analgesic agent injection by fluorescence.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising an analgesic and an imaging agent, wherein said analgesic is a transient vanilloid receptor 1 (TRPV1) antagonist.

2.-5. (canceled)

6. The composition of claim 1, wherein said TRPV1 antagonist is selected from the group consisting of capsazepine, ruthenium red, and derivatives and/or analogs thereof.

7. The composition of claim 1, wherein said analgesic is a nucleic acid encoding a polypeptide useful for treating pain.

8. The composition of claim 7, wherein said nucleic acid encoding a polypeptide useful for treating pain is present in a delivery vehicle.

9. The composition of claim 8, wherein said delivery vehicle is an adeno-associated virus vector.

10. The composition of claim 1, wherein said imaging agent is a non-neurotoxic imaging agent.

11. The composition of claim 1, wherein said imaging agent comprises gadolinium.

12. The composition of claim 1, wherein said composition further comprises a solubilizer.

13. The composition of claim 12, wherein said solubilizer is a non-neurotoxic solubilizer.

14. The composition of claim 12, where said solubilizer is a cyclodextrin.

15. The composition of claim 14, wherein said cyclodextrin is sulfobutyl ether β-cyclodextrin.

16. The composition of claim 1, wherein said composition is in the form of a pellet.

17. The composition of claim 1, wherein said composition is in the form of a gel.

18. The composition of claim 1, wherein said composition is in the form of a lyophilized powder.

19. A method for treating regional pain in a mammal, said method comprising:
injecting a composition comprising an analgesic and an imaging agent to a neural tissue of a mammal identified as having regional pain, wherein said analgesic is a transient vanilloid receptor 1 (TRPV1) antagonist;
wherein the regional pain is reduced.

20. A method for treating regional pain in a mammal, said method comprising:
injecting a composition comprising an analgesic and an imaging agent to a neural tissue of a mammal identified as having regional pain, wherein said analgesic is a transient vanilloid receptor 1 (TRPV1) antagonist;
wherein neurolysis of said neural tissue is induced.

21. The method of claim 19, wherein said mammal is a human.

22. The method of claim 19, wherein said injection comprises a spinal injection route.

23. The method of claim 22, wherein said spinal injection route is an intraganglionic (IG) injection or an injection to the subarachnoid space.

24. The method of claim 23, wherein said spinal injection route is an IG injection.

25. The method of claim 19, wherein said neural tissue is a ganglion.

26. The method of claim 25, wherein said ganglion is a dorsal root ganglion.

27. The method of claim 19, wherein said method further comprises monitoring said injection.

28. The method of claim 27, wherein said monitoring comprises an imaging technique selected from the group consisting of ultrasound, radiography, X-ray, computed tomography (CT), fluoroscopy, positron emission tomography, and magnetic resonance imaging (MRI).

29. The method of claim 28, wherein said imaging technique is MRI.

30. The method of claim 19, wherein said composition comprises from about 10 μL to about 500 μL.

31. The method of claim 30, wherein said composition comprises about 100 μL.

32. A kit comprising:
an analgesic;
an imaging agent;
a non-neurotoxic solubilizer;
a guide needle; and
a delivery needle.

33. The kit of claim 32, wherein said analgesic is lyophilized resiniferatoxin.

34. The kit of claim 32, wherein said non-neurotoxic solubilizer is a cyclodextrin.

35. The kit of claim 34, wherein said cyclodextrin is sulfobutyl ether β-cyclodextrin.
